(19) United States
(12) Patent Application Publication
(10) Pub. No.: US 2023/0210937 A1
(43) Pub. Date: Jul. 6, 2023

(54) COMPOSITIONS AND METHODS FOR THE TREATMENT AND/OR PROPHYLAXIS OF DIABETES AND OTHER CONDITIONS

(71) Applicant: LIFELOCK BIOSCIENCE SOLUTIONS INC., Vancouver (CA)

(72) Inventor: William David John CAMPBELL, Vancouver (CA)

(21) Appl. No.: 17/769,203

(22) PCT Filed: Oct. 19, 2020
(86) PCT No.: PCT/CA2020/051399

§ 371 Date: Apr. 14, 2022

Related U.S. Application Data
(60) Provisional application No. 62/916,919, filed on Oct. 18, 2019.

(51) Int. Cl. A61K 36/9066; A61K 31/05; A61K 31/353; A61K 31/6615; A61P 3/08

(52) U.S. Cl. CPC A61K 36/9066; A61K 31/05; A61K 31/353; A61K 31/6615; A61P 3/08

(57) ABSTRACT

A composition comprising curcumin, resveratrol, quercetin and inositol hexaphosphate. The composition has potential utility in treating high blood sugar, diabetes, heart disease and other chronic conditions including fibromyalgia and colitis, as well as cancer and arthritis. The composition also has potential utility in reducing inflammation, promoting weight loss, increasing energy levels, stimulating the immune system, increasing antioxidant levels, and preventing ageing.

### CROSS-REFERENCE TO RELATED APPLICATIONS

This application claims priority to, and the benefit of, U.S. provisional patent application No. 62/916,919 filed 18 Oct. 2019, the entirety of which is incorporated by reference herein.

### FIELD OF THE INVENTION

Some embodiments of the present invention relate to compositions and/or methods for the treatment and/or prophylaxis of diabetes or other chronic or inflammatory conditions. Some embodiments of the present invention relate to compositions and/or methods that may have other benefits such as increasing antioxidant levels, limiting or slowing the effects of ageing and/or treating chronic conditions such as colitis or fibromyalgia and/or treating cardiovascular disease and/or treating inflammation and/or arthritis.

### BACKGROUND

Type 2 diabetes is highly prevalent worldwide, affecting an estimated 500 million people. It is estimated that 30.3 million people in the US alone are affected, and that of these, 23.1 million are diagnosed and an estimated 7.2 million are undiagnosed. The disease is estimated to affect over 55 million people in the US alone by 2030.

In addition to direct suffering, type 2 diabetes also leads to heart, kidney and many other diseases. The associated health care costs are significant. Reported costs are estimated at US$327 billion for diagnosed U.S. population, with $237 billion in direct medical costs and $90 billion due to reduced productivity. Unreported costs are also believed to exist, relating to undiagnosed persons, prevention programs, over-the-counter costs due to medical issues related to type 2 diabetes, administrative costs for insurance claims, and reduced quality of life, as well as productivity losses of family members.

PAK-1 is a protein kinase involved in intracellular signaling pathways downstream of integrins and receptor-type kinases that plays an important role in cytoskeleton dynamics, in cell adhesion, migration, proliferation, apoptosis, mitosis, and in vesicle-mediated transport processes. The protein can directly phosphorylate BAD and protects cells against apoptosis, and is activated by interaction with CDC42 and RACI. PAK-1 functions as a GTPase effector that links the Rho-related GTPases CDC42 and RACI to the JNK MAP kinase pathway, and phosphorylates and activates MAP2K1, and thereby mediates activation of downstream MAP kinases. PAK-1 is involved in the reorganization of the actin cytoskeleton, actin stress fibers and of focal adhesion complexes, and phosphorylates the tubulin chaperone TBCB and thereby plays a role in the regulation of microtubule biogenesis and organization of the tubulin cytoskeleton. The protein also plays a role in the regulation of insulin secretion in response to elevated glucose levels, and is part of a ternary complex that contains PAK1, DVL1 and MUSK that is important for MUSK-dependent regulation of AChR clustering during the formation of the neuromuscular junction (NMJ).

PAK-1 activity is inhibited in cells undergoing apoptosis, potentially due to binding of CDC2L1 and CDC2L2. Other activities of PAK1 include: Phosphorylates MYL9/MLC2. Phosphorylates RAF1 at ‘Ser-338’ and ‘Ser-339’ resulting in: activation of RAF1, stimulation of RAF1 translocation to mitochondria, phosphorylation of BAD by RAF1, and RAF1 binding to BCL2. Phosphorylates SNAI1 at ‘Ser-246’ promoting its transcriptional repressor activity by increasing its accumulation in the nucleus. In podocytes, promotes NR3C2 nuclear localization. Required for atypical chemokine receptor ACKR2-induced phosphorylation of LIMK1 and cofilin (CFL1) and for the up-regulation of ACKR2 from endosomal compartment to cell membrane, increasing its efficiency in chemokine uptake and degradation. In synapses, seems to mediate the regulation of F-actin cluster formation performed by SHANK3, maybe through CFL1 phosphorylation and inactivation.

See for example Ahn et al., Diabetologia. 2016 October, 59(10): 2145-2155. doi:10.1007/s00125-016-4042-0, which is incorporated by reference herein in its entirety, “The p21-activated kinase (PAK1) is involved in diet-induced beta cell mass expansion and survival in mice and human islets. In that paper, the authors concluded that PAK1 deficiency may underlie an increased diabetic susceptibility, and suggested that discovery of ways to remediate glycaemic dysregulation via altering PAK1 or its downstream effectors offers promising opportunities for disease intervention. The authors hypothesized that human islets from type 2 diabetic donors are reportedly 80% deficient in the p21 (Cdc42/Rac)-activated kinase, PAK1. PAK1 is implicated in beta cell function and maintenance of beta cell mass. These authors questioned the mechanism(s) by which PAK1 deficiency potentially contributes to increased susceptibility to type 2 diabetes. To do this, non-diabetic human islets and INS 832/13 beta cells cultured under diabetogenic conditions (i.e. with specific cytokines or under glucolipotoxic[GLT] conditions) were evaluated for changes to PAK1 signalling. Combined effects of PAK1 deficiency with GLT stress were assessed using classic knockout (Pak1 (−/−)) mice fed a 45% energy from fat/palmitate-based, ‘western’ diet (WD). INS 832/13 cells overexpressing or depleted of PAK1 were also assessed for apoptosis and signalling changes. The results in this paper show exposure of non-diabetic human islets to diabetic stressors attenuated PAK1 protein levels, concurrent with increased caspase 3 cleavage. WD-fed Pak1 knockout mice exhibited fasting hyperglycaemia and severe glucose intolerance. These mice also failed to mount an insulin secretory response following acute glucose challenge, coinciding with a 43% loss of beta cell mass when compared with WD-fed wild-type mice. Pak1 knockout mice had fewer total beta cells per islet, coincident with decreased beta cell proliferation. In INS 832/13 beta cells, PAK1 deficiency combined with GLT exposure heightened beta cell death relative to either condition alone; PAK1 deficiency resulted in decreased extracellular signal-related kinase (ERK) and B cell lymphoma 2 (Bcl2) phosphorylation levels. Conversely, PAK1 overexpression prevented GLT-induced cell death.

See also Tunduguru et al., J Biol Chem. 2017 Nov. 17; 292(46):19034-19043. doi: 10.1074/jbc.M117.801340. Epub 2017 Sep. 25, which is incorporated by reference herein in its entirety, “The actin-related p41ARC subunit contributes to p21-activated kinase-1 (PAK1)-mediated glucose uptake into skeletal muscle cells” report that defects in translocation of the glucose transporter GLUT4 are associated with peripheral insulin resistance, preclinical diabetes, and progression to type 2 diabetes. GLUT4 recruitment to the plasma membrane of skeletal muscle cells requires F-actin remodeling. Insulin signaling in muscle requires p21-activated kinase-1 (PAK1), whose downstream signaling triggers actin remodeling, which promotes GLUT4 vesicle translocation and glucose uptake into skeletal muscle cells. Actin remodeling is a cyclic process, and although PAK1 is known to initiate changes to the cortical actin-binding protein cofilin to stimulate the depolymerizing arm of the cycle, how PAK1 might trigger the polymerizing arm of the cycle remains unresolved. Toward this, the authors investigated whether PAK1 contributes to the mechanisms involving the actin-binding and -polymerizing proteins neural Wiskott-Aldrich syndrome protein (N-WASP), cortactin, and ARP2/3 subunits. The authors found that the actin-polymerizing ARP2/3 subunit p41ARC is a PAK1 substrate in skeletal muscle cells. Moreover, co-immunoprecipitation experiments revealed that insulin stimulates p41ARC phosphorylation and increases its association with N-WASP coordinately with the associations of N-WASP with cortactin and actin. Importantly, all of these associations were ablated by the PAK inhibitor IPA3, suggesting that PAK1 activation lies upstream of these actin-polymerizing complexes. Using the N-WASP inhibitor wiskostatin, the authors further demonstrated that N-WASP is required for localized F-actin polymerization, GLUT4 vesicle translocation, and glucose uptake. These results expand the model of insulin-stimulated glucose uptake in skeletal muscle cells by implicating p41ARC as a new component of the insulin-signaling cascade and connecting PAK1 signaling to N-WASP-cortactin-mediated actin polymerization and GLUT4 vesicle translocation.

Curcumin (diferuloylmethane), is a polyphenol natural product of the plant Curcuma longa, is undergoing early clinical trials as a novel anticancer agent. However, the anticancer mechanism of curcumin remains to be elucidated. Recently, it has been shown that curcumin inhibits phosphorylation of p70S6kinase 1 (S6K1) and eukaryotic initiation factor 4E (eIF4E)-binding protein 1 (4E-BP1), two downstream effector molecules of the mammalian target of rapamycin complex 1(mTORC1) in numerous cancer cell lines. This study was designed to elucidate the underlying mechanism. Curcumin inhibits mTORC1 signaling not by inhibition of the upstream kinases, such as insulin-like growth factor 1receptor (IGF-IR) and phosphoinositide-dependent kinase 1 (PDK1). Curcumin inhibits mTORC1 signaling independently of protein phosphatase 2A (PP2A) or AMP-activated protein kinase AMPK-tuberous sclerosis complex (TSC). This is evidenced by the findings that curcumin is able to inhibit phosphorylation of S6K1 and 4E-BP1 in cells pretreated with PP2A inhibitor (okadaic acid) or AMPK inhibitor (compound C), or in cells expressing dominant negative (dn) PP2A, shRNA to PP2A-A subunit, or dn-AMPKA. Curcumin did not alter the TSC1/2 interaction. Knockout of TSC2 does not affect curcumin inhibition of mTOR signaling. Curcumin is able to dissociate raptor from mTOR, leading to inhibition of mTORC1 activity. See e.g. Cancer Res 2009; 69(3):1000-8.

Resveratrol mimics some of the effects of calorie restriction, in particular the rate of aging. Calorie restriction has a number of benefits including reducing metabolic rate. Reducing metabolic rate allows mitochondria (power stations in cells) not to work so hard or burn as much fuel, so less toxins and waste products are produced including free radicals. Insulin production is also lowered which reduces the risks of both diabetes and cancer. IGF-1 levels are also lowered resulting in inhibition of rapid multiplication of cancer cells. An important discovery reported in “Nature” showed that in three species, administering resveratrol increased longevity by stimulating the survival defence enzyme Sir which in turn stimulates the production of sirtuins, the same survival defence hormones produced when animals are on restricted calorie diets. The detrimental effects of a high calorie diet were negated at the genetic level due to improved insulin sensitivity, reduced IgF-1 levels and increased mitochondrial numbers. (See e.g. Nature 2006; 444:337-342). In another study, resveratrol and curcumin together seemed to prevent tumor cell growth and induce cancer cell death in neuroblastomas by activating the p53 gene pathway. The downregulation of PI3K/Akt/mTOR signaling pathways may be an important mediator in resveratrol-induced apoptosis in glioma cells.

Quercetin is a flavonoid with anticancer activity. Quercetin is a mitochondrial ATPase and phosphodiesterase inhibitor. It inhibits PI3-kinase activity and slightly inhibits PIP kinase activity. Quercetin has antiproliferative effects on cancer cell lines, reduces cancer cell growth via type II estrogen receptors, and arrests human leukemic T cells in late G1 phase of the cell cycle. Quercetin may also inhibit fatty acid synthase activity. Quercetin is a potent anti-tumor agent having anti-inflammatory and anti-oxidant properties. Several health benefits have been reported for quercetin including protection against various diseases such as osteoporosis, certain forms of cancer, pulmonary and cardiovascular disease as well as protection against aging. The ability of quercetin to scavenge highly reactive oxygen species such as peroxynitrate and the hydroxyl radical is suggested to be involved in its beneficial health effects. (see e.g. European Journal of Pharmacology, 2008, May 13, 325-37).

Inositol hexaphosphate (IP6) is a naturally occurring phytochemical, found in abundance in cereals, legumes and other high-fiber-content diets. IP6 has shown promising efficacy against a wide range of cancers. Its anti-cancer activity involves anti-proliferative, pro-apoptotic and anti-metastatic effects. Both matrix metalloproteinases (MMPs) and their tissue inhibitors (TIMPs) are implicated in tumor growth, metastasis, and angiogenesis. Phorbol-12-myristate 13-acetate (PMA) is a well-known inflammatory stimulator and tumor promoter that activates PKC and increases the invasiveness of various types of cancer cells by activating MMPs. One study examined the influence of IP6 on the expression of selected MMPs, i.e., MMP-1, -2, -3, -9, 10, -13 and their TIMP-1 and -2 in unstimulated and PMA-stimulated colon cancer cell line Caco-2. Quantification of genes expression in Caco-2 cells treated with 100 ng/mL of PMA, 2.5 mM of IP6 and both for 6 and 12 h was carried out using real time QRT-PCR technique. Stimulation of cells with PMA resulted in an up-expression of MMP-2, MMP-3, MMP-9, MMP-10, MMP-13 and TIMP-1 mRNAs and decrease in MMP-1 gene expression. The quantity of TIMP-2 transcript was reduced by PMA. A significant decrease in MMP-2, MMP-3, MMP-10, MMP-13, and TIMP-1 expression in response to IP6 was observed. IP6 down-regulated MMP-9 transcription induced by PMA and decreased the level of both MMP-2 and MMP-3 mRNAs in PMA-stimulated cells. Caco-2 treated with both PMA and IP6 showed a significant decrease in MMP-1 expression in comparison to PMA-stimulated cells. The results of this study showed that PMA can modulate MMP and TIMP genes transcription in colon cancer cells Caco-2. IP6 exerts an influence of basal mRNA expression of some MMPs and their tissue inhibitors and down-regulates MMP-1, MMP-2, MMP-3 and MMP-9 in cells treated with PMA. IP6 could be an effective anti-metastatic agent that suppresses expression of MMP genes at transcription level.

The foregoing examples of the related art and limitations related thereto are intended to be illustrative and not exclusive. Other limitations of the related art will become apparent to those of skill in the art upon a reading of the specification and a study of the drawings.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

One aspect provides a formulation containing curcumin, resveratrol, quercetin and inositol hexaphosphate. The formulation can be supplied as an oral dosage form containing between about 50 and about 600 mg each of curcumin, resveratrol, quercetin and inositol hexaphosphate. The level of each compound present in the dosage form can be the same or can be different. The oral dosage form can contain between about 100 and about 400 mg of each of curcumin, resveratrol, quercetin and inositol hexaphosphate. The oral dosage form can contain approximately 100 mg each of curcumin, resveratrol, quercetin and inositol hexaphosphate. The formulation or oral dosage form can be combined with a pharmaceutically acceptable carrier or excipient. The oral dosage form can be a tablet or a capsule.

One aspect provides a method of treating diabetes or high blood sugar levels by administering to a patient in need a formulation or an oral dosage form as described herein. One aspect provides a method of treating a chronic condition by administering to a patient in need a formulation or an oral dosage form as described herein. The chronic condition can be diabetes, heart disease, fibromyalgia or colitis. One aspect provides a method of preventing, treating and/or ameliorating cancer by administering to a patient in need a formulation or an oral dosage form as described herein. One aspect provides a method of preventing aging by administering to a subject a formulation or an oral dosage form as described herein. One aspect provides a method of promoting weight loss, enhancing immune system function, increasing antioxidant levels and/or increasing energy levels by administering to a subject a formulation or an oral dosage form as described herein. The patient or subject can receive a daily dose of between about 50 and about 600 mg of each, optionally between about 100 and about 400 mg of each, of curcumin, resveratrol, quercetin and inositol hexaphosphate. The patient or subject can receive a daily dosage regime of about 1 mg/kg to about 10 mg/kg, optionally between about 1 mg/kg to about 5 mg/kg, optionally between about 1 mg/kg to about 2 mg/kg of each of curcumin, resveratrol, quercetin and inositol hexaphosphate. The daily dose of each compound can be the same or can be different.

The patient or subject can be a mammalian subject, including a human.

In addition to the exemplary aspects and embodiments described above, further aspects and embodiments will become apparent by reference to the drawings and by study of the following detailed descriptions.

### BRIEF DESCRIPTION OF THE DRAWINGS

Exemplary embodiments are illustrated in referenced figures of the drawings. It is intended that the embodiments and FIGURES disclosed herein are to be considered illustrative rather than restrictive.

FIG. 1 shows an overview of the putative mechanism of mTOR action.

### DETAILED DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

The inventor has now determined that a biotherapeutic containing natural ingredients can be prepared from a combination of resveratrol, curcumin, quercetin and inositol hexaphosphate. Such composition has efficacy in the prophylaxis and/or treatment of diabetes and other chronic conditions. Such composition may also have efficacy in slowing or delaying the aging process. Such composition may boost glucose metabolism and surprisingly increases activity of the PAK1 protein, while type 2 diabetics are e.g. about 80% deficient in PAK1 protein. Such composition may also exhibit an anti-inflammatory effect to reduce age-related diseases and chronic conditions. Without being bound by theory, such composition may exhibit anti-aging effects by slowing the nutrient sensing mechanism, mimicking calorie restriction, and/or acting as an antioxidant to reduce DNA damage and lower predisposition to cancer and/or enhancing antioxidant levels within the body.

Resveratrol (3,5,4′-trihydroxy-trans-stilbene) is a stilbenoid, which is a type of natural phenol. The compound is produced by some plants in response to injury or when the plant is under attack by pathogens. Resveratrol has the following structure (1):

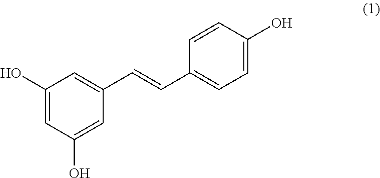 [SMILES: Oc1ccc(/C=C/c2cc(O)cc(O)c2)cc1]

Without being bound by theory, resveratrol may have efficacy in increasing longevity in animals, and may yield improved insulin sensitivity. Resveratrol is also believed to induce a protein that suppresses tumor growth. Specifically, longevity in animals is increased by resveratrol by stimulating a survival defense enzyme, Sir that in turn stimulates production of Sirtuins. Improved insulin sensitivity is also an observed effect of this compound. Together with curcumin, there is a demonstrated ability to suppress cancer tumor growth by inducing the p53 tumor suppressor protein activation pathway.

Curcumin ((1E,6E)-1,7-Bis(4-hydroxy-3-methoxyphenyl)hepta-1,6-diene-3,5-dione) is a chemical produced by Curcuma longa plants, and is the principal curcuminoid found in turmeric, which is a member of the ginger family. Curcumin is believed to inhibit mTOR signaling and to mimic the effect of calorie restriction, which may slow down the aging process. Specifically, curcumin has been shown to inhibit S6K1 and 4E-BP1, two downstream effectors of m-TOR. Curcumin is also able to dissociate raptor from TOR inhibiting TOR1 activity. This mimics the effects seen with calorie restriction and therefore is believed to act to slow down the aging process. Curcumin has the following structure, and can exist in either an enol form (left) (2) or a keto form (right) (3):

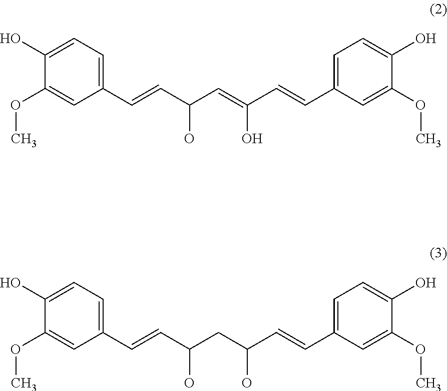 [SMILES: COc1cc(/C=C/C(O)=C/C(O)/C=C/c2ccc(O)c(OC)c2)ccc1O ; COc1cc(/C=C/C(O)CC(O)/C=C/c2ccc(O)c(OC)c2)ccc1O]

Quercetin (2-(3,4-dihydroxyphenyl)-3,5,7-trihydroxy-4H-chromen-4-one) is a plant flavonol from the flavonoid group of polyphenols, and is found in many fruit, vegetables leaves and grains, including red onions and kale. Quercetin is believed to inhibit P13 kinase and slow the growth of cancer cells, and also to have an anti-inflammatory and anti-oxidant effect that is important in maintaining heart health. Quercetin has the following structure (4):

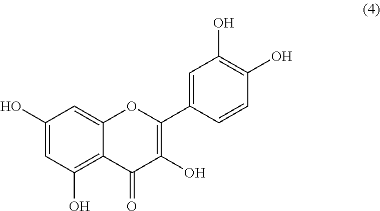 [SMILES: O=c1c(O)c(-c2ccc(O)c(O)c2)oc2cc(O)cc(O)c12]

Inositol hexaphosphate or phytic acid ((1R,2S,3R,4R,5S,6s)-cyclohexane-1,2,3,4,5,6-hexayl hexakis[dihydrogen (phosphate)]) is a six-fold dihydrogenphosphate ester of inositol, and is the principal storage form of phosphorus in plant seeds. Inositol hexaphosphate is believed to act as an effective anti-metastatic agent by suppressing expression of MMP genes at the transcription level. Inositol hexaphosphate has the following structure (5):

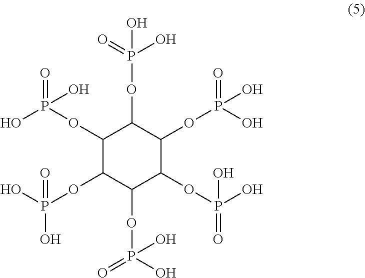 [SMILES: O=P(O)(O)OC1C(OP(=O)(O)O)C(OP(=O)(O)O)C(OP(=O)(O)O)C(OP(=O)(O)O)C1OP(=O)(O)O]

The inventor has found that by combining four components that have been previously shown to possess a number of positive effects, i.e. resveratrol, curcumin, quercetin and inositol hexaphosphate, unexpected synergies and effects are observed. The resulting composition may yield a number of positive effects that may contribute to treatment of chronic conditions and slowing down of the aging process. Without being bound by theory, it is believed that the composition acts at the molecular level to affect certain cell signaling events. The composition may achieve its desirable effects by reducing inflammation, as chronic inflammation is a source of biochemical damage that contributes to age-related diseases or chronic conditions. Such diseases may include diabetes or high blood sugar, cardiac disease, colitis, fibromyalgia and the like, as well as inflammation and conditions such as arthritis that may relate to inflammation. The composition may also exert preventive, therapeutic and/or prophylactic effects on other disorders such as cancer. The composition may also stimulate the immune system, increase antioxidant levels, increase energy levels, and/or promote weight loss. Aspects of the invention can relate to methods of treating any of the foregoing conditions or promoting such beneficial effects by administering a composition as described herein.

The composition can be administered in any desirable form or manner, and may be combined with any desired acceptable pharmaceutical carriers, excipients and the like. In some embodiments, the composition is formulated for oral administration. In some embodiments, the resveratrol, curcumin, quercetin and inositol hexaphosphate are administered together, e.g. in a single dosage form. In some embodiments, the resveratrol, curcumin, quercetin and inositol hexaphosphate are administered separately, e.g. in four separate dosage forms. In some embodiments, various combinations of two or more of the resveratrol, curcumin, quercetin and inositol hexaphosphate are administered in separate dosage forms. The separate dosage forms may be taken together or may be administered at different times.

The composition may be formulated into any desired dosage form. In some embodiments, the composition is formulated as an oral dosage form containing between about 50 and about 600 mg of each of the resveratrol, curcumin, quercetin and inositol hexaphosphate, including any value or subrange therebetween, e.g. about 75, 100, 125, 150, 175, 200, 225, 250, 275, 300, 325, 350, 375, 400, 425, 450, 475, 500, 525, 550, or 575 mg of each of the resveratrol, curcumin, quercetin and inositol hexaphosphate. In some embodiments, the oral dosage form is a tablet or capsule. The amount of each of the resveratrol, curcumin, quercetin and inositol hexaphosphate present in the oral dosage form may be the same or may be different.

In some embodiments, the composition is administered at a daily dose of about 50 to about 600 mg of each of the resveratrol, curcumin, quercetin and inositol hexaphosphate, whether administered separately, together, or in any desired combination, and including any value or subrange therebetween, e.g. a daily dose of about 75, 100, 125, 150, 175, 200, 225, 250, 275, 300, 325, 350, 375, 400, 425, 450, 475, 500, 525, 550, or 575 mg of each of the resveratrol, curcumin, quercetin and inositol hexaphosphate. The daily dose of each of the resveratrol, curcumin, quercetin and inositol hexaphosphate may be the same or may be different.

In some embodiments, the composition is administered at a daily dosage rate of about 1 mg/kg/day to about 10 mg/kg/day of each of the resveratrol, curcumin, quercetin and inositol hexaphosphate, whether administered separately, together or in any desired combination, and including any value or subrange therebetween, e.g. a daily dosage rate of about 1.25, 1.50, 1.75, 2.00, 2.25, 2.50, 2.75, 3.00, 3.25, 3.50, 3.75, 4.00, 4.25, 4.50, 4.75, 5.00, 5.25, 5.50, 5.75, 6.00, 6.25, 6.50, 6.75, 7.00, 7.25, 7.50, 7.75, 8.00, 8.25, 8.50, 8.75, 9.00, 9.25, 9.50 or 9.75 mg/kg/day of each of the resveratrol, curcumin, quercetin and inositol hexaphosphate. The daily dose rate of each of the resveratrol, curcumin, quercetin and inositol hexaphosphate may be the same or may be different.

In one example embodiment, the composition is administered orally in a capsule form. In one example embodiment, the capsule contains approximately 100 mg of resveratrol, 100 mg of curcumin, 100 mg of quercetin, and 100 mg of inositol hexaphosphate. In one example embodiment, a subject is administered one capsule per day orally. In another example embodiment, a subject is administered two, three or four capsules per day. In one example embodiment, the composition is administered to provide a daily dose of approximately 1-2 mg/kg of resveratrol, 1-2 mg/kg of curcumin, 1-2 mg/kg of quercetin, and 1-2 mg/kg of inositol hexaphosphate.

The composition may be administered to a mammalian subject or patient. The mammalian subject may be a human, cat, dog, horse, cow, sheep, pig or other mammal.

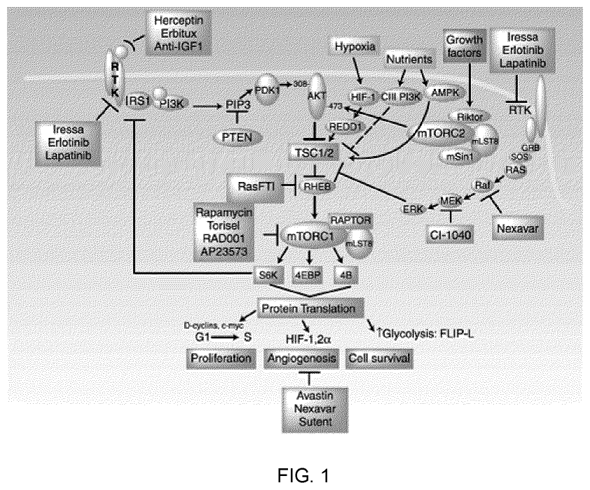

Without being bound by theory, FIG. 1 indicates a potential mechanism of action of the composition comprising the resveratrol, curcumin, quercetin and inositol hexaphosphate via the mTOR (mammalian target of rapamycin) protein.

### EXAMPLES

Specific embodiments are further described with reference to the following examples, which are intended to be illustrative and not limiting in nature.

### Example 1.0—Effect of Composition on Protein Expression Levels in Human Cell Line

In a study using human cells (Jurkat cell line), the inventor measured the effect of the composition containing resveratrol, curcumin, quercetin and inositol hexaphosphate on the expression levels of 800 different proteins using antibody microarrays.

General observations of the results are that there was an increase in three tumor suppressor proteins, showing a powerful anti-cancer effect. The highest up-regulated protein due to the effect of the composition was PAK-1. A decrease in PAK-1 kinase is seen in diabetics, suggesting that administration of the composition may be effective in the prophylaxis and/or treatment of diabetes. An increase in regulation of glucose metabolism significant for diabetes was also observed. Also observed was a decrease in heat shock proteins reflecting a lowering of the effect of environmental stress from infection and inflammation, which is a positive effect. Heat shock proteins are increased under different types of environmental stress such as infection and inflammation and are usually increased under conditions of stress.

More specifically, several positive effects were observed but the most significant was an increase (3.75 fold) in the level of PAK1, a cell signalling protein. Patients with Type 2 diabetes are in some cases 80% deficient in PAK1 suggesting that individuals with PAK1 deficiency would benefit from the composition. PAK1 is important in the regulation of glucose and insulin metabolism. Thus, these results suggest that administration of the composition would be effective in the prophylaxis and/or treatment of diabetes.

There was also significant up regulation of three tumor suppressor proteins, p53 tumor suppressor protein, DAPK1a tumor suppressor candidate and p27 cyclin dependent kinase inhibitor.

Conditions of the tested samples are shown in Table 1 below. Each of the compounds was tested individually, and a combination of all four compounds was also tested. The concentration of each compound in sample 6 was 0.9 mg/mL.

TABLE 1
Samples Tested for Protein Expression.

| | | | Treatment | Concentration |
|---|---|---|---|---|
| No. | Control | Sample Name | Information | (mg/ml) |
| 1 | Y | Control- No | No treatment | 1.7 |
| | | Treatment | | |
| 2 | N | Curcumin | Curcumin, | 1.6 |
| | | | 25 μM, 24 hr | |
| 3 | N | Quercetin | Quercetin, | 1.8 |
| | | | 100 μM, 24 hr | |
| 4 | N | Resveratrol | Resveratrol, | 1.3 |
| | | | 25 μM, 24 hr | |
| 5 | N | IP6 - Phytic Acid | IP6-Phytic Acid, | 1.8 |
| | | | 2 mM, 24 hr | |
| 6 | N | All 4 Compounds | All 4 Compounds, | 0.9 (each) |
| | | | 24 hr | |

To perform the analyses, 50 μg of lysate protein from each sample are covalently labeled with a fluorescent dye. Free dye molecules are then removed at the completion of labeling reactions by gel filtration. After blocking non-specific binding sites on the array, an incubation chamber is mounted onto the microarray to permit the loading of 2 samples (normally one control and one matching treated sample) side by side on the same chip and prevent mixing of the samples. Following sample incubation, unbound proteins are washed away. Each array produces a pair of 16-bit images, which are captured with a Perkin-Elmer ScanArray Reader laser array scanner (Waltham, Mass.).

Signal quantification is performed with ImaGene 8.0 from BioDiscovery (El Segundo, Calif.) with predetermined settings for spot segmentation and background correction. The background-corrected raw intensity data are logarithmically transformed with base 2. Since Z normalization in general displays greater stability as a result of examining where each signal falls in the overall distribution of values within a given sample, as opposed to adjusting all of the signals in a sample by a single common value, Z scores are calculated by subtracting the overall average intensity of all spots within a sample from the raw intensity for each spot, and dividing it by the standard deviations (SD) of all of the measured intensities within each sample (Cheadle et al., Analysis of microarray data using Z score transformation. Journal of Molecular Diagnostics 5, 73-81, 2003).

Z ratios are further calculated by taking the difference between the averages of the observed protein Z scores and dividing by the SD of all of the differences for that particular comparison. Calculated Z ratios have the advantage that they can be used in multiple comparisons without further reference to the individual conditional standard deviations by which they were derived. A Z ratio of 1.2 to 1.5 is inferred as significant. For convenience, the changes in spot intensity between control and treatment samples are also expressed as the percent change from control (% CFC) using globally normalized data.

A summary of the results for the genes that were up-regulated in the sample treated with all four compounds (Sample 6) are provided in Table 2 below, together with the UniProtKB accession number for each gene.

TABLE 2
Summary of genes upregulated by treatment
of Jurkat cell line with composition.

| | | Accession |
|---|---|---|
| Gene Name | Z Ratio | No. |
| p21-activated kinase 1 (alpha) (serine/threonine- | 3.79 | Q13153 |
| protein kinase PAK 1) | | |
| Docking protein 2 | 2.67 | O60496 |
| Eukaryotic translation initiation factor 2 alpha | 2.46 | P05198 |
| Dual specificity MAP kinase protein phosphatase | 2.42 | Q05923 |
| Epidermal growth factor receptor-tyrosine kinase | 2.39 | P00533 |
| Protein-serine phosphatase 5 - catalytic subunit | 2.34 | P53041 |
| (PPT) | | |
| DNA damage-inducible transcript 3 protein | 2.33 | P35639 |
| Catenin (cadherin-associated protein) beta 1 | 2.30 | P35222 |
| ErbB2 (Neu) receptor-tyrosine kinase | 2.23 | P04626 |
| Tumor suppressor protein p53 (antigenNY-CO-13) | 2.22 | P04637 |
| CDK5 regulatory subunit p25 | 2.03 | Q15078 |
| Kinase homologous to SPS1/STE20 (MAP kinase | 2.02 | Q9Y4K4 |
| kinase kinase protein-serine kinase 5 (MEKKK5) | | |
| p27 cyclin-dependent kinase inhibitor 1B | 1.97 | P46527 |
| Signal transducer and activator of transcription 2 | 1.85 | P52630 |
| Protein-serine phosphatase 4 - regulatory | 1.80 | Q8TF05 |
| subunit (PPX/A′2) | | |
| Calcium/calmodulin-dependent protein-serine | 1.75 | Q8IU85 |
| kinase 1 delta | | |
| Glycogen synthase-serine kinase 3 alpha | 1.70 | P49840 |
| ETS domain-containing protein Elk-1 | 1.68 | P19419 |
| Protein-serine kinase suppressor of Ras 1 | 1.65 | Q8IVT5 |
| Lymphocyte-specific protein-tyrosine kinase | 1.62 | P06239 |
| Epidermal growth factor receptor-tyrosine kinase | 1.60 | P00533 |
| Mammalian STE20-like protein-serine kinase 1 | 1.59 | Q13043 |
| (KRS2) | | |
| Protein-serine phosphatase 2B - catalytic | 1.59 | Q08209 |
| subunit - alpha isoform | | |
| MAP kinase-interacting protein-serine kinase 2 | 1.56 | Q9HBH9 |
| (calmodulin-activated) | | |
| ErbB2 (Neu) receptor-tyrosine kinase | 1.55 | P04626 |
| NF-kappa-B p50 nuclear transcription factor | 1.52 | P19838 |
| p18 INK4c cyclin-dependent kinase inhibitor | 1.47 | P42773 |
| Jun N-terminus protein-serine kinase (stress- | 1.44 | P45983 |
| activated protein kinase (SAPK)) 1/2/3 | | |
| Kit/Steel factor receptor-tyrosine kinase | 1.43 | P10721 |
| A-Raf proto-oncogene serine/threonine-protein | 1.43 | P10398 |
| kinase | | |
| MAPK/ERK protein-serine kinase 7 (MKK7) | 1.38 | O14733 |
| Germinal centre protein-serine kinase | 1.36 | Q12851 |
| Tumor suppressor protein p53 (antigenNY-CO-13) | 1.35 | P04637 |
| Protein kinase C-related protein-serine kinase 1 | 1.29 | Q16512 |
| MAPK/ERK kinase kinase 2 | 1.25 | Q9Y2U5 |
| Protein-tyrosine kinase 2 | 1.23 | Q14289 |
| Mixed-lineage protein-serine kinase 3 | 1.21 | Q16584 |
| Epithelial cell adhesion molecule | 1.20 | P16422 |

A description of the function of the genes identified by the study other than PAK1 that are believed to be supportive of a synergistic effect of the composition and their Z ratio when all four compounds were administered together (Sample 6) follows below.

### 53 Q06187—BTK_HUMAN

Bruton's agammaglobulinemia tyrosine kinase, Z ratio 1.31. Non-receptor tyrosine kinase indispensable for B lymphocyte development, differentiation and signaling. Binding of antigen to the B-cell antigen receptor (BCR) triggers signaling that ultimately leads to B-cell activation. After BCR engagement and activation at the plasma membrane, phosphorylates PLCG2 at several sites, igniting the downstream signaling pathway through calcium mobilization, followed by activation of the protein kinase C (PKC) family members. PLCG2 phosphorylation is performed in close cooperation with the adapter protein B-cell linker protein BLNK. BTK acts as a platform to bring together a diverse array of signaling proteins and is implicated in cytokine receptor signaling pathways. Plays an important role in the function of immune cells of innate as well as adaptive immunity, as a component of the Toll-like receptors (TLR) pathway. The TLR pathway acts as a primary surveillance system for the detection of pathogens and are crucial to the activation of host defense. Especially, is a critical molecule in regulating TLR9 activation in splenic B-cells. Within the TLR pathway, induces tyrosine phosphorylation of TIRAP which leads to TIRAP degradation. BTK plays also a critical role in transcription regulation. Induces the activity of NF-kappa-B, which is involved in regulating the expression of hundreds of genes. BTK is involved on the signaling pathway linking TLR8 and TLR9 to NF-kappa-B. Transiently phosphorylates transcription factor GTF2I on tyrosine residues in response to BCR. GTF2I then translocates to the nucleus to bind regulatory enhancer elements to modulate gene expression. ARID3A and NFAT are other transcriptional target of BTK. BTK is required for the formation of functional ARID3A DNA-binding complexes. There is however no evidence that BTK itself binds directly to DNA. BTK has a dual role in the regulation of apoptosis.

### 60 Q8IU85—KCC1D_HUMAN

Calcium/calmodulin-dependent protein-serine kinase 1 delta, Z ratio 1.75.

Calcium/calmodulin-dependent protein kinase that operates in the calcium-triggered CaMKK-CaMK1 signaling cascade and, upon calcium influx, activates CREB-dependent gene transcription, regulates calcium-mediated granulocyte function and respiratory burst and promotes basal dendritic growth of hippocampal neurons. In neutrophil cells, required for cytokine-induced proliferative responses and activation of the respiratory burst. Activates the transcription factor CREB1 in hippocampal neuron nuclei. May play a role in apoptosis of erythroleukemia cells. In vitro, phosphorylates transcription factor CREM isoform Beta.

### 82 P35222—CTNB1_HUMAN

Catenin (cadherin-associated protein) beta 1, Z ratio 2.30. Key downstream component of the canonical Wnt signaling pathway. In the absence of Wnt, forms a complex with AXIN1, AXIN2, APC, CSNK1A1 and GSK3B that promotes phosphorylation on N-terminal Ser and Thr residues and ubiquitination of CTNNB1 via BTRC and its subsequent degradation by the proteasome. In the presence of Wnt ligand, CTNNB1 is not ubiquitinated and accumulates in the nucleus, where it acts as a coactivator for transcription factors of the TCF/LEF family, leading to activate Wnt responsive genes. Involved in the regulation of cell adhesion. Acts as a negative regulator of centrosome cohesion. Involved in the CDK2/PTPN6/CTNNB1/CEACAM1 pathway of insulin internalization. Blocks anoikis of malignant kidney and intestinal epithelial cells and promotes their anchorage-independent growth by down-regulating DAPK2. Disrupts PML function and PML-NB formation by inhibiting RANBP2-mediated sumoylation of PML (see e.g. Lillehoj et al., Biochim. Biophys. Acta 1773:1028-1038 (2007); Weiske et al., Proc. Natl. Acad. Sci. U.S.A. 104:20344-20349 (2007); Bahmanyar et al., Genes Dev. 22:91-105 (2008); Li et al., J. Biol. Chem. 284:2012-2022 (2009); Fiset et al., Cell. Signal. 23:911-919 (2011); Genovese et al., Cell Cycle 11:2206-2215 (2012); Yu et al., EMBO Rep. 13:750-758 (2012); Satow et al., Gastroenterology 142:572-581 (2012)). Promotes neurogenesis by maintaining sympathetic neuroblasts within the cell cycle (By similarity).

### 104 P06493—CDK1_HUMAN (UNIQUE)

Cyclin-dependent protein-serine kinase ½, Z ratio −1.46. Plays a key role in the control of the eukaryotic cell cycle by modulating the centrosome cycle as well as mitotic onset; promotes G2-M transition, and regulates G1 progress and G1-S transition via association with multiple interphase cyclins. Required in higher cells for entry into S-phase and mitosis. Phosphorylates PARVA/actopaxin, APC, AMPH, APC, BARD1, Bcl-xL/BCL2L1, BRCA2, CALD1, CASP8, CDC7, CDC20, CDC25A, CDC25C, CC2D1A, CSNK2 proteins/CKII, FZR1/CDH1, CDK7, CEBPB, CHAMP1, DMD/dystrophin, EEF1 proteins/EF-1, EZH2, KIF11/EG5, EGFR, FANCG, FOS, GFAP, GOLGA2/GM130, GRASP1, UBE2A/hHR6A, HIST1H1 proteins/histone H1, HMGA1, HIVEP3/KRC, LMNA, LMNB, LMNC, LBR, LATS1, MAP1B, MAP4, MARCKS, MCM2, MCM4, MKLP1, MYB, NEFH, NFIC, NPC/nuclear pore complex, PITPNM1/NIR2, NPM1, NCL, NUCKS1, NPM1/numatrin, ORC1, PRKAR2A, EEF1E1/p18, EIF3F/p47, p53/TP53, NONO/p54NRB, PAPOLA, PLEC/plectin, RB1, UL40/R2, RAB4A, RAP1GAP, RCC1, RPS6KB1/S6K1, KHDRBS1/SAM68, ESPL1, SKI, BIRC5/survivin, STIP1, TEX14, beta-tubulins, MAPT/TAU, NEDD1, VIM/vimentin, TK1, FOXO1, RUNX1/AML1, SIRT2 and RUNX2. CDK1/CDC2-cyclin-B controls pronuclear union in interphase fertilized eggs. Essential for early stages of embryonic development. During G2 and early mitosis, CDC25A/B/C-mediated dephosphorylation activates CDK1/cyclin complexes which phosphorylate several substrates that trigger at least centrosome separation, Golgi dynamics, nuclear envelope breakdown and chromosome condensation. Once chromosomes are condensed and aligned at the metaphase plate, CDK1 activity is switched off by WEE1- and PKMYT1-mediated phosphorylation to allow sister chromatid separation, chromosome decondensation, reformation of the nuclear envelope and cytokinesis. Inactivated by PKR/EIF2AK2- and WEE1-mediated phosphorylation upon DNA damage to stop cell cycle and genome replication at the G2 checkpoint thus facilitating DNA repair. Reactivated after successful DNA repair through WIP1-dependent signaling leading to CDC25A/B/C-mediated dephosphorylation and restoring cell cycle progression. In proliferating cells, CDK1-mediated FOXO1 phosphorylation at the G2-M phase represses FOXO1 interaction with 14-3-3 proteins and thereby promotes FOXO1 nuclear accumulation and transcription factor activity, leading to cell death of postmitotic neurons. The phosphorylation of beta-tubulins regulates microtubule dynamics during mitosis. NEDD1 phosphorylation promotes PLK1-mediated NEDD1 phosphorylation and subsequent targeting of the gamma-tubulin ring complex (gTuRC) to the centrosome, an important step for spindle formation. In addition, CC2D1A phosphorylation regulates CC2D1A spindle pole localization and association with SCC1/RAD21 and centriole cohesion during mitosis. The phosphorylation of Bcl-xL/BCL2L1 after prolonged G2 arrest upon DNA damage triggers apoptosis. In contrast, CASP8 phosphorylation during mitosis prevents its activation by proteolysis and subsequent apoptosis. This phosphorylation occurs in cancer cell lines, as well as in primary breast tissues and lymphocytes. EZH2 phosphorylation promotes H3K27me3 maintenance and epigenetic gene silencing. CALD1 phosphorylation promotes Schwann cell migration during peripheral nerve regeneration.

### 181 O060496—DOK2_HUMAN

Docking protein 2, Z ratio 2.57. DOK proteins are enzymatically inert adaptor or scaffolding proteins. They provide a docking platform for the assembly of multimolecular signaling complexes. DOK2 may modulate the cellular proliferation induced by IL-4, as well as IL-2 and IL-3. May be involved in modulating Bcr-Abl signaling. Attenuates EGF-stimulated MAP kinase activation.

### 182 P52799—EFNB2_HUMAN

Docking protein 2, Z ratio 2.67. Cell surface transmembrane ligand for Eph receptors, a family of receptor tyrosine kinases which are crucial for migration, repulsion and adhesion during neuronal, vascular and epithelial development. Binds promiscuously Eph receptors residing on adjacent cells, leading to contact-dependent bidirectional signaling into neighboring cells. The signaling pathway downstream of the receptor is referred to as forward signaling while the signaling pathway downstream of the ephrin ligand is referred to as reverse signaling. Binds to receptor tyrosine kinase including EPHA4, EPHA3 and EPHB4. Together with EPHB4 plays a central role in heart morphogenesis and angiogenesis through regulation of cell adhesion and cell migration. EPHB4-mediated forward signaling controls cellular repulsion and segregation from EFNB2-expressing cells. May play a role in constraining the orientation of longitudinally projecting axons.

### 183 P00533—EGFR_HUMAN

DAP kinase-related apoptosis-inducing protein-serine kinase 2 (STK17B), Z ratio 2.23. Receptor tyrosine kinase binding ligands of the EGF family and activating several signaling cascades to convert extracellular cues into appropriate cellular responses. Known ligands include EGF, TGFA/TGF-alpha, amphiregulin, epigen/EPGN, BTC/betacellulin, epiregulin/EREG and HBEGF/heparin-binding EGF. Ligand binding triggers receptor homo- and/or heterodimerization and autophosphorylation on key cytoplasmic residues. The phosphorylated receptor recruits adapter proteins like GRB2 which in turn activates complex downstream signaling cascades. Activates at least 4 major downstream signaling cascades including the RAS-RAF-MEK-ERK, PI3 kinase-AKT, PLCgamma-PKC and STATs modules. May also activate the NF-kappa-B signaling cascade. Also directly phosphorylates other proteins like RGS16, activating its GTPase activity and probably coupling the EGF receptor signaling to the G protein-coupled receptor signaling. Also phosphorylates MUC1 and increases its interaction with SRC and CTNNB1/beta-catenin.

### 186 P52799—EFNB2_HUMAN

EPH-related receptor tyrosine kinase ligand 5, Z ratio 1.84. Cell surface transmembrane ligand for Eph receptors, a family of receptor tyrosine kinases which are crucial for migration, repulsion and adhesion during neuronal, vascular and epithelial development. Binds promiscuously Eph receptors residing on adjacent cells, leading to contact-dependent bidirectional signaling into neighboring cells. The signaling pathway downstream of the receptor is referred to as forward signaling while the signaling pathway downstream of the ephrin ligand is referred to as reverse signaling. Binds to receptor tyrosine kinase including EPHA4, EPHA3 and EPHB4. Together with EPHB4 plays a central role in heart morphogenesis and angiogenesis through regulation of cell adhesion and cell migration. EPHB4-mediated forward signaling controls cellular repulsion and segregation from EFNB2-expressing cells. May play a role in constraining the orientation of longitudinally projecting axons.

### 190 P00533—EGFR_HUMAN

Epidermal growth factor receptor-tyrosine kinase, Z ratio 3.35. Receptor tyrosine kinase binding ligands of the EGF family and activating several signaling cascades to convert extracellular cues into appropriate cellular responses. Known ligands include EGF, TGFA/TGF-alpha, amphiregulin, epigen/EPGN, BTC/betacellulin, epiregulin/EREG and HBEGF/heparin-binding EGF. Ligand binding triggers receptor homo- and/or heterodimerization and autophosphorylation on key cytoplasmic residues. The phosphorylated receptor recruits adapter proteins like GRB2 which in turn activates complex downstream signaling cascades. Activates at least 4 major downstream signaling cascades including the RAS-RAF-MEK-ERK, PI3 kinase-AKT, PLCgamma-PKC and STATs modules. May also activate the NF-kappa-B signaling cascade. Also directly phosphorylates other proteins like RGS16, activating its GTPase activity and probably coupling the EGF receptor signaling to the G protein-coupled receptor signaling. Also phosphorylates MUC1 and increases its interaction with SRC and CTNNB1/beta-catenin. Isoform 2 (P00533-2) may act as an antagonist of EGF action.

### 202 Q04637—IF4G1_HUMAN

Eukaryotic translation initiation factor 4 gamma 1, Z ratio. Eukaryotic translation initiation factor 4 gamma 1, Z ratio 2.54. Component of the protein complex elF4F, which is involved in the recognition of the mRNA cap, ATP-dependent unwinding of 5′-terminal secondary structure and recruitment of mRNA to the ribosome.

### 203 P19419—ELK1_HUMAN

ETS domain-containing protein Elk-1, Z ratio 4.29. Stimulates transcription. Binds to purine-rich DNA sequences. Can form a ternary complex with the serum response factor and the ETS and SRF motifs of the fos serum response element.

### 209 P04626—ERBB2_HUMAN

ErbB2 (Neu) receptor-tyrosine kinase, Z ratio 4.81. Protein tyrosine kinase that is part of several cell surface receptor complexes, but that apparently needs a coreceptor for ligand binding. Essential component of a neuregulin-receptor complex, although neuregulins do not interact with it alone. GP30 is a potential ligand for this receptor. Regulates outgrowth and stabilization of peripheral microtubules (MTs). Upon ERBB2 activation, the MEMO1-RHOA-DIAPH1 signaling pathway elicits the phosphorylation and thus the inhibition of GSK3B at cell membrane. This prevents the phosphorylation of APC and CLASP2, allowing its association with the cell membrane. In turn, membrane-bound APC allows the localization of MACF1 to the cell membrane, which is required for microtubule capture and stabilization.

In the nucleus is involved in transcriptional regulation. Associates with the 5′-TCAAATTC-3′ sequence in the PTGS2/COX-2 promoter and activates its transcription. Implicated in transcriptional activation of CDKN1A; the function involves STAT3 and SRC. Involved in the transcription of rRNA genes by RNA Pol I and enhances protein synthesis and cell growth.

### 276 P49840—GSK3A_HUMAN

Glycogen synthase-serine kinase 3 alpha, Z ratio 1.70. Constitutively active protein kinase that acts as a negative regulator in the hormonal control of glucose homeostasis, Wnt signaling and regulation of transcription factors and microtubules, by phosphorylating and inactivating glycogen synthase (GYS1 or GYS2), CTNNB1/beta-catenin, APC and AXIN1. Requires primed phosphorylation of the majority of its substrates. Contributes to insulin regulation of glycogen synthesis by phosphorylating and inhibiting GYS1 activity and hence glycogen synthesis. Regulates glycogen metabolism in liver, but not in muscle. May also mediate the development of insulin resistance by regulating activation of transcription factors. In Wnt signaling, regulates the level and transcriptional activity of nuclear CTNNB1/beta-catenin. Facilitates amyloid precursor protein (APP) processing and the generation of APP-derived amyloid plaques found in Alzheimer disease. May be involved in the regulation of replication in pancreatic beta-cells. Is necessary for the establishment of neuronal polarity and axon outgrowth. Through phosphorylation of the anti-apoptotic protein MCL1, may control cell apoptosis in response to growth factors deprivation.

### 397 Q16667—CDKN3_HUMAN

Cyclin-dependent kinase associated phosphatase (CDK inhibitor 3, CIP2), Z ratio 1.13. May play a role in cell cycle regulation. Dual specificity phosphatase active toward substrates containing either phosphotyrosine or phosphoserine residues. Dephosphorylates CDK2 at ‘Thr-160’ in a cyclin-dependent manner.

### 503 P19838—NFKB1_HUMAN

NF-kappa-B p50 nuclear transcription factor, Z ratio 1.52. NF-kappa-B is a pleiotropic transcription factor present in almost all cell types and is the endpoint of a series of signal transduction events that are initiated by a vast array of stimuli related to many biological processes such as inflammation, immunity, differentiation, cell growth, tumorigenesis and apoptosis. NF-kappa-B is a homo- or heterodimeric complex formed by the Rel-like domain-containing proteins RELA/p65, RELB, NFKB1/p105, NFKB1/p50, REL and NFKB2/p52 and the heterodimeric p65-p50 complex appears to be most abundant one. The dimers bind at kappa-B sites in the DNA of their target genes and the individual dimers have distinct preferences for different kappa-B sites that they can bind with distinguishable affinity and specificity. Different dimer combinations act as transcriptional activators or repressors, respectively. NF-kappa-B is controlled by various mechanisms of post-translational modification and subcellular compartmentalization as well as by interactions with other cofactors or corepressors. NF-kappa-B complexes are held in the cytoplasm in an inactive state complexed with members of the NF-kappa-B inhibitor (I-kappa-B) family. In a conventional activation pathway, I-kappa-B is phosphorylated by I-kappa-B kinases (IKKs) in response to different activators, subsequently degraded thus liberating the active NF-kappa-B complex which translocates to the nucleus. NF-kappa-B heterodimeric p65-p50 and RelB-p50 complexes are transcriptional activators. The NF-kappa-B p50-p50 homodimer is a transcriptional repressor, but can act as a transcriptional activator when associated with BCL3. NFKB1 appears to have dual functions such as cytoplasmic retention of attached NF-kappa-B proteins by p105 and generation of p50 by a cotranslational processing. The proteasome-mediated process ensures the production of both p50 and p105 and preserves their independent function, although processing of NFKB1/p105 also appears to occur post-translationally. p50 binds to the kappa-B consensus sequence 5′-GGRNNYYCC-3′, located in the enhancer region of genes involved in immune response and acute phase reactions. In a complex with MAP3K8, NFKB1/p105 represses MAP3K8-induced MAPK signaling; active MAP3K8 is released by proteasome-dependent degradation of NFKB1/p105.

### 526 Q16539—MK14_HUMAN UNIQUE

Mitogen-activated protein-serine kinase p38 alpha, Z ratio 2.09. Serine/threonine kinase which acts as an essential component of the MAP kinase signal transduction pathway. MAPK14 is one of the four p38 MAPKs which play an important role in the cascades of cellular responses evoked by extracellular stimuli such as proinflammatory cytokines or physical stress leading to direct activation of transcription factors. Accordingly, p38 MAPKs phosphorylate a broad range of proteins and it has been estimated that they may have approximately 200 to 300 substrates each. Some of the targets are downstream kinases which are activated through phosphorylation and further phosphorylate additional targets. RPS6KA5/MSK1 and RPS6KA4/MSK2 can directly phosphorylate and activate transcription factors such as CREB1, ATF1, the NF-kappa-B isoform RELA/NFKB3, STAT1 and STAT3, but can also phosphorylate histone H3 and the nucleosomal protein HMGN1. RPS6KA5/MSK1 and RPS6KA4/MSK2 play important roles in the rapid induction of immediate-early genes in response to stress or mitogenic stimuli, either by inducing chromatin remodeling or by recruiting the transcription machinery. On the other hand, two other kinase targets, MAPKAPK2/MK2 and MAPKAPK3/MK3, participate in the control of gene expression mostly at the post-transcriptional level, by phosphorylating ZFP36 (tristetraprolin) and ELAVL1, and by regulating EEF2K, which is important for the elongation of mRNA during translation. MKNK1/MNK1 and MKNK2/MNK2, two other kinases activated by p38 MAPKs, regulate protein synthesis by phosphorylating the initiation factor EIF4E2. MAPK14 interacts also with casein kinase II, leading to its activation through autophosphorylation and further phosphorylation of TP53/p53. In the cytoplasm, the p38 MAPK pathway is an important regulator of protein turnover. For example, CFLAR is an inhibitor of TNF-induced apoptosis whose proteasome-mediated degradation is regulated by p38 MAPK phosphorylation. In a similar way, MAPK14 phosphorylates the ubiquitin ligase SIAH2, regulating its activity towards EGLN3. MAPK14 may also inhibit the lysosomal degradation pathway of autophagy by interfering with the intracellular trafficking of the transmembrane protein ATG9. Another function of MAPK14 is to regulate the endocytosis of membrane receptors by different mechanisms that impinge on the small GTPase RAB5A. In addition, clathrin-mediated EGFR internalization induced by inflammatory cytokines and UV irradiation depends on MAPK14-mediated phosphorylation of EGFR itself as well as of RAB5A effectors. Ectodomain shedding of transmembrane proteins is regulated by p38 MAPKs as well. In response to inflammatory stimuli, p38 MAPKs phosphorylate the membrane-associated metalloprotease ADAM17. Such phosphorylation is required for ADAM17-mediated ectodomain shedding of TGF-alpha family ligands, which results in the activation of EGFR signaling and cell proliferation. Another p38 MAPK substrate is FGFR1. FGFR1 can be translocated from the extracellular space into the cytosol and nucleus of target cells, and regulates processes such as rRNA synthesis and cell growth. FGFR1 translocation requires p38 MAPK activation. In the nucleus, many transcription factors are phosphorylated and activated by p38 MAPKs in response to different stimuli. Classical examples include ATF1, ATF2, ATF6, ELK1, PTPRH, DDIT3, TP53/p53 and MEF2C and MEF2A. The p38 MAPKs are emerging as important modulators of gene expression by regulating chromatin modifiers and remodelers. The promoters of several genes involved in the inflammatory response, such as IL6, IL8 and IL12B, display a p38 MAPK-dependent enrichment of histone H3 phosphorylation on ‘Ser-10’ (H3S10ph) in LPS-stimulated myeloid cells. This phosphorylation enhances the accessibility of the cryptic NF-kappa-B-binding sites marking promoters for increased NF-kappa-B recruitment. Phosphorylates CDC25B and CDC25C which is required for binding to 14-3-3 proteins and leads to initiation of a G2 delay after ultraviolet radiation. Phosphorylates TIAR following DNA damage, releasing TIAR from GADD45A mRNA and preventing mRNA degradation. The p38 MAPKs may also have kinase-independent roles, which are thought to be due to the binding to targets in the absence of phosphorylation. Protein O-Glc-N-acylation catalyzed by the OGT is regulated by MAPK14, and, although OGT does not seem to be phosphorylated by MAPK14, their interaction increases upon MAPK14 activation induced by glucose deprivation. This interaction may regulate OGT activity by recruiting it to specific targets such as neurofilament H, stimulating its O-Glc-N-acylation. Required in mid-fetal development for the growth of embryo-derived blood vessels in the labyrinth layer of the placenta. Also plays an essential role in developmental and stress-induced erythropoiesis, through regulation of EPO gene expression. Isoform MXI2 activation is stimulated by mitogens and oxidative stress and only poorly phosphorylates ELK1 and ATF2. Isoform EXIP may play a role in the early onset of apoptosis. Phosphorylates S100A9 at ‘Thr-113’.

### 531 P53778—MK12_HUMAN UNIQUE

Mitogen-activated protein-serine kinase p38 gamma (MAPK12), Z ratio 1.63. Serine/threonine kinase which acts as an essential component of the MAP kinase signal transduction pathway. MAPK12 is one of the four p38 MAPKs which play an important role in the cascades of cellular responses evoked by extracellular stimuli such as proinflammatory cytokines or physical stress leading to direct activation of transcription factors such as ELK1 and ATF2. Accordingly, p38 MAPKs phosphorylate a broad range of proteins and it has been estimated that they may have approximately 200 to 300 substrates each. Some of the targets are downstream kinases such as MAPKAPK2, which are activated through phosphorylation and further phosphorylate additional targets. Plays a role in myoblast differentiation and also in the down-regulation of cyclin D1 in response to hypoxia in adrenal cells suggesting MAPK12 may inhibit cell proliferation while promoting differentiation. Phosphorylates DLG1. Following osmotic shock, MAPK12 in the cell nucleus increases its association with nuclear DLG1, thereby causing dissociation of DLG1-SFPQ complexes. This function is independent of its catalytic activity and could affect mRNA processing and/or gene transcription to aid cell adaptation to osmolarity changes in the environment. Regulates UV-induced checkpoint signaling and repair of UV-induced DNA damage and G2 arrest after gamma-radiation exposure. MAPK12 is involved in the regulation of SLC2A1 expression and basal glucose uptake in L6 myotubes; and negatively regulates SLC2A4 expression and contraction-mediated glucose uptake in adult skeletal muscle. C-Jun (JUN) phosphorylation is stimulated by MAPK14 and inhibited by MAPK12, leading to a distinct AP-1 regulation. MAPK12 is required for the normal kinetochore localization of PLK1, prevents chromosomal instability and supports mitotic cell viability. MAPK12-signaling is also positively regulating the expansion of transient amplifying myogenic precursor cells during muscle growth and regeneration.

### 532 P04637—P53_HUMAN

Tumor suppressor protein p53 (antigenNY—CO-13), Z ratio 2.22. Acts as a tumor suppressor in many tumor types; induces growth arrest or apoptosis depending on the physiological circumstances and cell type. Involved in cell cycle regulation as a trans-activator that acts to negatively regulate cell division by controlling a set of genes required for this process. One of the activated genes is an inhibitor of cyclin-dependent kinases. Apoptosis induction seems to be mediated either by stimulation of BAX and FAS antigen expression, or by repression of Bcl-2 expression. In cooperation with mitochondrial PPIF is involved in activating oxidative stress-induced necrosis; the function is largely independent of transcription. Induces the transcription of long intergenic non-coding RNA p21 (lincRNA-p21) and lincRNA-Mkln1. LincRNA-p21 participates in TP53-dependent transcriptional repression leading to apoptosis and seem to have to effect on cell-cycle regulation. Implicated in Notch signaling cross-over. Prevents CDK7 kinase activity when associated to CAK complex in response to DNA damage, thus stopping cell cycle progression. Isoform 2 enhances the transactivation activity of isoform 1 from some but not all TP53-inducible promoters. Isoform 4 suppresses transactivation activity and impairs growth suppression mediated by isoform 1. Isoform 7 inhibits isoform 1-mediated apoptosis. Regulates the circadian clock by repressing CLOCK-ARNTL/BMAL1-mediated transcriptional activation of PER2.

### 678 P78352—DLG4_HUMAN UNIQUE

Disks large homolog 4, Z ratio 2.53. Interacts with the cytoplasmic tail of NMDA receptor subunits and shaker-type potassium channels. Required for synaptic plasticity associated with NMDA receptor signaling. Overexpression or depletion of DLG4 changes the ratio of excitatory to inhibitory synapses in hippocampal neurons. May reduce the amplitude of ASIC3 acid-evoked currents by retaining the channel intracellularly. May regulate the intracellular trafficking of ADR1B (By similarity).

### 703 P10398—ARAF_HUMAN

A-Raf proto-oncogene serine/threonine-protein kinase, Z ratio 1.43. Involved in the transduction of mitogenic signals from the cell membrane to the nucleus. May also regulate the TOR signaling cascade. 1 Publication Isoform 2: Serves as a positive regulator of myogenic differentiation by inducing cell cycle arrest, the expression of myogenin and other muscle-specific proteins, and myotube formation

Other genes of interest include 192 (EGFR) (Epidermal growth factor receptor-tyrosine kinase), Z ratio 2.39, 193 (EGFR) (Epidermal growth factor receptor-tyrosine kinase) (unique), Z ratio 2.52, 238 (FAK) (Focal adhesion protein-tyrosine kinase) (minus), Z ratio −3.26 and 536 (PN158 Tumor suppressor protein p53 (antigenNY—CO-13), Z ratio 2.50).

### Example 2.0—Demonstration of Anti-Oxidant Effect

A Pharmanex BioPhotonic Scanner was used to noninvasively measure carotenoid antioxidant levels in skin. Using the instrument, in 90 seconds a score can be generated that can identify the risk of free radical attacks, by simply placing the palm of the subject's hand in front of the Scanner's LED light.

Ten subjects were evaluated over the course of two months using the BioPhonic scanner as an objective measuring tool. Each subject was given a certified skin carotenoid score at day zero, establishing a reliable biomarker of each subject's overall antioxidant health status. Each subject was then tested 60 days later to assess for increases in antioxidants. Best results assessed by increase in antioxidant levels were observed in subjects consuming 400 mg per day of each of resveratrol, curcumin, quercetin and inositol hexaphosphate.

### Example 3.0—Relief of Diabetes and Inflammation

A male subject experienced type 2 diabetes with high levels of blood sugar and cholesterol, as well as cardiovascular disease and inflammation. The subject was being treated with numerous medications for those conditions. The subject consumed 200 mg each of resveratrol, curcumin, quercetin and inositol hexaphosphate twice daily (once in the morning and once in the evening, for a total of 400 mg each of resveratrol, curcumin, quercetin and inositol hexaphosphate per day). The subject began to notice positive results after 4-5 months and his treatment with metformin was discontinued as his type 2 diabetes was found to have resolved. Over the course of treatment, the subject also lost 58 pounds of excess weight and experienced an 80% decrease in inflammation, and has subsequently not experienced negative cardiovascular symptoms.

### Example 4.0—Relief of Long Term Conditions

A female subject experienced fibromyalgia and colitis for more than 15 years, which various treatments had failed to resolve. The subject consumed 100 mg each of resveratrol, curcumin, quercetin and inositol hexaphosphate with each meal (for a total of 300 mg per day of each of resveratrol, curcumin, quercetin and inositol hexaphosphate). The subject experienced relief of belly cramps commencing on day 1 of the treatment, and has found that urgent trips to the bathroom have subsided and overall body pain has dropped to a level 3 from the usual 6-8 (out of 10 being the most pain).

### Example 5.0—Relief of Arthritis

A female subject suffered from arthritis pain for several years. She consumed 200 mg each of resveratrol, curcumin, quercetin and inositol hexaphosphate with morning and evening meals daily (for a total of 400 mg per day each of resveratrol, curcumin, quercetin and inositol hexaphosphate) and experienced weight loss and relief of arthritis pain. The subject also experienced benefits of an enhanced immune system and increased energy levels while consuming this composition. When she discontinued consumption of the composition for a few months, she experienced return of arthritis pain.

### Example 6.0—Relief of High Blood Sugar Levels

A female subject who was unable to control high blood sugar levels consumed 100 mg each of resveratrol, curcumin, quercetin and inositol hexaphosphate with three meals per day (for a total of 300 mg per day each of resveratrol, curcumin, quercetin and inositol hexaphosphate). After two weeks, the subject experienced subjectively enhanced wellbeing, and after two months of consuming the composition the subject's blood sugar levels were observed to be within normal ranges. The subject also expressed having higher energy levels as well.

The foregoing examples demonstrate that administration of a combination of resveratrol, curcumin, quercetin and inositol hexaphosphate can increase expression of PAK-1, which supports a therapeutic and/or prophylactic role for the composition in for example the treatment of diabetes or controlling high blood sugar levels. Testing in human subjects establishes that administration of the composition can help bring high blood sugar levels and type 2 diabetes under control and/or resolve such conditions, as well as bringing relief of other chronic conditions such as fibromyalgia and colitis, as well as arthritis and cardiac disease. Further, subjects administered the composition experienced increased levels of antioxidants, weight loss, increased energy levels, enhanced immune system function, and a decrease in inflammation.

While a number of exemplary aspects and embodiments have been discussed above, those of skill in the art will recognize certain modifications, permutations, additions and sub-combinations thereof. It is therefore intended that any claims hereafter introduced are interpreted to include all such modifications, permutations, additions and sub-combinations as are consistent with the broadest interpretation of the specification as a whole.

All references mentioned in this specification are incorporated by reference herein in their entireties.

1. A formulation comprising curcumin, resveratrol, quercetin, and inositol hexaphosphate.

2. An oral dosage form comprising the formulation as defined in claim 1, wherein the oral dosage form comprises between:
about 50 and about 600 mg of curcumin;
about 50 and about 600 mg of resveratrol;
about 50 and about 600 mg of quercetin; and
about 50 and about 600 mg of inositol hexaphosphate.

3. An oral dosage form comprising the formulation as defined in claim 1, wherein the oral dosage form comprises between:
about 100 and about 400 mg of curcumin;
about 100 and about 400 mg of resveratrol;
about 100 and about 400 mg of quercetin; and
about 100 and about 400 mg of inositol hexaphosphate.

4. An oral dosage form as defined in claim 1, wherein the oral dosage form comprises:
approximately 100 mg of curcumin;
approximately 100 mg of resveratrol;
approximately 100 mg of quercetin; and
approximately 100 mg of inositol hexaphosphate.

5. A formulation or oral dosage form as defined in claim 1, further comprising a pharmaceutically acceptable carrier or excipient.

6. An oral dosage form as defined in claim 1 that is a tablet or a capsule.

7. A method of treating diabetes or high blood sugar levels, comprising administering to a patient in need a therapeutically effective amount of a formulation or an oral dosage form as defined in claim 1.

8. A method of treating a chronic condition, comprising administering to a patient in need a therapeutically effective amount of a formulation or an oral dosage form as defined in claim 1.

9. A method as defined in claim 8, wherein the chronic condition is diabetes, heart disease, fibromyalgia or colitis.

10. A method of treating inflammation, comprising administering to a patient in need a therapeutically effective amount of a formulation or an oral dosage form as defined in claim 1.

11. A method of treating arthritis, comprising administering to a patient in need a therapeutically effective amount of a formulation as defined in claim 1.

12. A method of preventing, treating and/or ameliorating cancer, comprising administering to a patient in need a formulation as defined in claim 1.

13. A method of promoting weight loss, increasing energy levels, enhancing immune system function or increasing antioxidant levels, comprising administering to a patient in need a formulation as defined in claim 1.

14. A method of preventing aging, comprising administering to a subject a formulation as defined in claim 1.

15. A method as defined in claim 7, wherein the subject or patient is administered a daily dose of between:
about 50 and about 600 mg of curcumin;
about 50 and about 600 mg of resveratrol;
about 50 and about 600 mg of quercetin; and
about 50 and about 600 mg of inositol hexaphosphate.

16. A method as defined in claim 7, wherein the subject or patient is administered a daily dose of between:
about 100 to about 400 mg of curcumin;
about 100 and about 400 mg of resveratrol;
about 100 and about 400 mg of quercetin; and
about 100 and about 400 mg of inositol hexaphosphate.

17. A method as defined in claim 7, wherein the subject or patient is administered a daily dose of:
about 1 mg/kg to about 10 mg/kg of curcumin;
about 1 mg/kg to about 10 mg/kg of resveratrol;
about 1 mg/kg to about 10 mg/kg of quercetin; and
about 1 mg/kg to about 10 mg/kg of inositol hexaphosphate.

18. A method as defined in claim 7, wherein the subject or patient is administered a daily dose of:
about 1 mg/kg to about 5 mg/kg of curcumin;
about 1 mg/kg to about 5 mg/kg of resveratrol;
about 1 mg/kg to about 5 mg/kg of quercetin; and
about 1 mg/kg to about 5 mg/kg of inositol hexaphosphate.

19. A method as defined in claim 7, wherein the subject or patient is administered a daily dose of:
about 1 mg/kg to about 2 mg/kg of curcumin;
about 1 mg/kg to about 2 mg/kg of resveratrol;
about 1 mg/kg to about 2 mg/kg of quercetin; and
about 1 mg/kg to about 2 mg/kg of inositol hexaphosphate.

20. (canceled)

21. A method as defined in claim 7, wherein the patient is a mammalian patient, optionally a human patient.
